(19) United States
(12) Patent Application Publication
(10) Pub. No.: US 2026/0193186 A1
(43) Pub. Date: Jul. 9, 2026

(54) PYRIDINIUM SALTS-BASED CHEMICALS THAT INCLUDE SULFUR

(71) Applicant: Saudi Arabian Oil Company, Dhahran (SA)

(72) Inventors: Muhammad Imran Ul-haq, Dhahran (SA); Mohammad Shoaib, Dhahran (SA)

(73) Assignee: Saudi Arabian Oil Company, Dhahran (SA)

(21) Appl. No.: 19/014,081

(22) Filed: Jan. 8, 2025

(51) Int. Cl. C07D 213/20; C23F 11/14

(52) U.S. Cl. CPC C07D 213/20; C23F 11/14

(57) ABSTRACT

Chemical compounds that include a pyridinium-based compound functionalized by including a sulfur-containing substituent are provided. Also provided are materials and formulations including such pyridinium-based compounds. Also provided are corrosion-resistant substrates and films including such pyridinium-based compounds. Also provided are processes for inhibiting corrosion of a metallic surface using the formulations and processes of acidizing a subsurface formation using the pyridinium-based compounds.

### TECHNICAL FIELD

The present disclosure relates to chemical compounds and, more specifically, to pyridinium-based chemical compounds.

### BACKGROUND

Corrosion is an issue for many materials when they interact with their environments over time. For example, the presence of species such as H₂S, CO₂, organic acids, and brine solutions in produced oil may create a corrosive environment for transportation pipelines and oil processing units in an oil and gas facility. Specifically, when CO₂ and H₂S are dissolved in water, these species may behave like weak acids and promote the corrosion of steel, thus resulting in damage to the internal walls of the transportation pipelines and oil processing units and causing leaks that will increase the maintenance time and costs associated with the oil and gas processing. Many conventional compounds may be used in corrosion inhibitors and corrosion-resistant films in order to reduce corrosion of surfaces. However, these conventional compounds are often toxic and non-biodegradable. Additionally, there is a relatively high cost associated with the production of these conventional compounds. Further, these conventional compounds do not sufficiently resist the corrosive effects present in the wet environment (i.e., an environment rich in H₂S) often present in crude oil processing facilities. As such, new compounds are needed for use in corrosion inhibitors and corrosion-resistant films.

### SUMMARY

Described herein are pyridinium-based compounds as well as materials that include these pyridinium-based compounds. Such pyridinium-based compounds may include a pyridinium functional group, where the nitrogen atom of the pyridinium is further functionalized by including a group that includes at least one additional sulfur atom, as is described herein. The pyridinium-based compounds described herein may reduce corrosion of metallic surfaces, such as carbon steel. For example, such pyridinium-based compounds may be present in, for example, and without limitation, well fluids, formulations comprising water, and corrosion-resistant substrates. In some embodiments, such pyridinium-based compounds may be utilized as emulsifiers to form emulsions. Additionally, such pyridinium-based compounds may be utilized in methods for inhibiting corrosion by contact with a substrate such as a pipe, or in acidizing processes. The presence of pyridinium-based compounds may, in some embodiments, perform as well or better than conventional compounds known to inhibit corrosion.

According to one or more embodiments of the present disclosure, a pyridinium-based compound may have the structure of Chemical Structure #1, described hereinbelow, or a salt thereof. One or both of X may be —SH or Y may be —S—. R¹ may be a C₁-C₁₈ alkyl group, a C₁-C₁₈ hydroxyl alkyl group, a C₁-C₁₈ alkenyl group, a C₁-C₁₈ internal alkynyl group, a C₁-C₁₈ acryl group, a C₁-C₁₈ cycloalkyl group, or a C₁-C₁₈ functional alkyl group. R_{A}, R_{B}, R_{C}, R_{D}, and R_{E} may each independently be chosen from hydrogen, a C₁-C₁₈ alkyl group, a C₁-C₁₈ hydroxyl alkyl group, a C₁-C₁₈ alkenyl group, a C₁-C₁₈ alkynyl group, a C₁-C₁₈ acryl group, a C₁-C₁₈ cycloalkyl group, or a C₁-C₁₈ functional alkyl group.

According to one or more additional embodiments of the present disclosure, a material may comprise a pyridinium-based compound. The pyridinium-based compound may have the structure of Chemical Structure #1, described hereinbelow, or a salt thereof. At least one of X or Y may be N. R¹ may be a C₁-C₁₈ alkyl group, a C₁-C₁₈ hydroxyl alkyl group, a C₁-C₁₈ alkenyl group, a C₁-C₁₈ internal alkynyl group, a C₁-C₁₈ acryl group, a C₁-C₁₈ cycloalkyl group, or a C₁-C₁₈ functional alkyl group. R_{A}, R_{B}, R_{C}, R_{D}, and R_{E} may each independently be chosen from hydrogen, a C₁-C₁₈ alkyl group, a C₁-C₁₈ hydroxyl alkyl group, a C₁-C₁₈ alkenyl group, a C₁-C₁₈ alkynyl group, a C₁-C₁₈ acryl group, a C₁-C₁₈ cycloalkyl group, or a C₁-C₁₈ functional alkyl group.

According to one or more yet additional embodiments of the present disclosure, a corrosion-resistant substrate may comprise a substrate comprising a first surface and a corrosion-resistant film positioned on at least a portion of the first surface of the substrate. The first surface may be metal or metal oxide. The corrosion-resistant film may be solid. The corrosion-resistant film may comprises a pyridinium-based compound that may have the structure of Chemical Structure #1, described hereinbelow, or a salt thereof. One or both of X may be —SH or Y may be —S—. R¹ may be a C₁-C₁₈ alkyl group, a C₁-C₁₈ hydroxyl alkyl group, a C₁-C₁₈ alkenyl group, a C₁-C₁₈ internal alkynyl group, a C₁-C₁₈ acryl group, a C₁-C₁₈ cycloalkyl group, or a C₁-C₁₈ functional alkyl group. R_{A}, R_{B}, R_{C}, R_{D}, and R_{E} may each independently be chosen from hydrogen, a C₁-C₁₈ alkyl group, a C₁-C₁₈ hydroxyl alkyl group, a C₁-C₁₈ alkenyl group, a C₁-C₁₈ alkynyl group, a C₁-C₁₈ acryl group, a C₁-C₁₈ cycloalkyl group, or a C₁-C₁₈ functional alkyl group.

According to one or more yet additional embodiments of the present disclosure, a subsurface formation may be acidized by a method that may comprise injecting an acidic treatment fluid into a subsurface formation through a tubing of an extraction well, thereby exposing at least a portion of the tubing to an acidic environment. At least a portion of the tubing may contacted by a pyridinium-based compound. The pyridinium-based compound may have the structure of Chemical Structure #1, described hereinbelow, or a salt thereof. One or both of X may be —SH or Y may be —S—. R¹ may be a C₁-C₁₈ alkyl group, a C₁-C₁₈ hydroxyl alkyl group, a C₁-C₁₈ alkenyl group, a C₁-C₁₈ internal alkynyl group, a C₁-C₁₈ acryl group, a C₁-C₁₈ cycloalkyl group, or a C₁-C₁₈ functional alkyl group. R_{A}, R_{B}, R_{C}, R_{D}, and R_{E} may each independently be chosen from hydrogen, a C₁-C₁₈ alkyl group, a C₁-C₁₈ hydroxyl alkyl group, a C₁-C₁₈ alkenyl group, a C₁-C₁₈ alkynyl group, a C₁-C₁₈ acryl group, a C₁-C₁₈ cycloalkyl group, or a C₁-C₁₈ functional alkyl group.

These and other embodiments are described in more detail in the detailed description. It is to be understood that both the foregoing general description and the following detailed description present embodiments of the presently disclosed technology, and are intended to provide an overview or framework for understanding the nature and character of the presently disclosed technology as it is claimed. The accompanying drawings are included to provide a further understanding of the presently disclosed technology and are incorporated into and constitute a part of this specification. The drawings illustrate various embodiments and, together with the description, serve to explain the principles and operations of the presently disclosed technology. Additionally, the drawings and descriptions are meant to be merely illustrative, and are not intended to limit the scope of the claims in any manner.

### BRIEF DESCRIPTION OF THE DRAWINGS

The following detailed description of specific embodiments of the present disclosure can be best understood when read in conjunction with the following drawings, where like structure is indicated with like reference numerals and wherein:

FIG. 1 schematically depicts a cross-sectional view of a substrate comprising a corrosion-resistant film, according to one or more embodiments shown and described herein;

FIG. 2 schematically depicts a cross-sectional view cut in the axial direction of a metal pipe comprising a corrosion-resistant film, according to one or more embodiments shown and described herein; and

FIG. 3 schematically depicts a hydrocarbon extraction well, according to one or more embodiments shown and described herein.

### DETAILED DESCRIPTION

One or more embodiments of the present disclosure are directed to pyridinium-based compounds. The pyridinium-based compounds described herein may generally be described by Chemical Structure #1, where X is —SH (a thiol group), Y is —S— (a sulfur atom), or both X is —SH and Y is —S—. According to some embodiments, the pyridinium-based compounds may be cations, as shown in Chemical Structure #1, where the N atom of the pyridinium is shown as having a positive charge. In additional embodiments, the pyridinium-based compounds may be salts, where an anion (not shown) is bonded with the compound of Chemical Structure #1. For example, anions may include F, Cl, Br, or I, but are not particularly limited herein. In Chemical Structure #1, one or both of X may be —SH or Y may be —S—. In embodiments, if X is not —SH but Y is —S—, X may be —CH₃, —NH₂, or —OH. In embodiments, if X is —SH but Y is not —S—, Y may be —CH₂, —NH, or —O—.

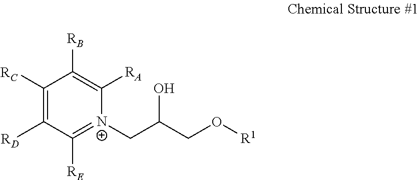

Referring to Chemical Structure #1, as well as other chemical structures describe herein, the general structure includes R¹, R_{A}, R_{B}, R_{C}, R_{D}, and R_{E} that each represent various functional groups. Unless specified otherwise, discussion herein of R groups may refer to both the pyridinium-based compounds represented by Chemical Structure #1. R¹ may be a C₁-C₁₈ alkyl group, a C₁-C₁₈ hydroxyl alkyl group, a C₁-C₁₈ alkenyl group, a C₁-C₁₈ internal alkynyl group, a C₁-C₁₈ acryl group, a C₁-C₁₈ cycloalkyl group, or a C₁-C₁₈ functional alkyl group. R_{A}, R_{B}, R_{C}, R_{D}, and R_{E} may each independently chosen from hydrogen, a C₁-C₁₈ alkyl group, a C₁-C₁₈ hydroxyl alkyl group, a C₁-C₁₈ alkenyl group, a C₁-C₁₈ alkynyl group, a C₁-C₁₈ acryl group, a C₁-C₁₈ cycloalkyl group, or a C₁-C₁₈ functional alkyl group. Without being bound by a theory, it is believed that one or more of R¹, R_{A}, R_{B}, R_{C}, R_{D}, and R_{E} having a relatively long carbon chain moiety allows the pyridinium-based compound to better adhere to a metal surface, such as tubing. Further, if the carbon chain moiety has greater than 18 carbon atoms, there may be an increased risk of the pyridinium-based compound has poor adherence to metal substrates.

In one or more embodiments, the term “functional group” or “group” may refer to a substituent or moiety that is present in the pyridinium-based compound. For example, when the disclosure states that R¹ may be a methyl group, the methyl group (—CH₃) replaces R¹ of the general structure of the 1-(2-hydroxyalkyl)pyridinium compound, where the carbon atom of the methyl group is now bonded to the carbon atom of the 1-(2-hydroxyalkyl)pyridinium compound to which R¹ bonded.

As described herein, moieties may be defined by the number of carbon atoms included in the moiety, such as Cₓ-C_{y}, where x is the least number of carbon atoms and y is the greatest number of carbon atoms contemplated. For example, C₁-C₁₈ describes a moiety that has from 1 to 18 carbon atoms.

In some embodiments, R¹, R_{A}, R_{B}, R_{C}, R_{D}, and R_{E} may each independently be a C₁-C₁₈ alkyl group. The term “alkyl group” refers to a functional group that only contains carbon and hydrogen atoms where the carbon atoms and hydrogen atoms are only connected by single bonds. In some embodiments, R¹, R_{A}, R_{B}, R_{C}, R_{D}, and R_{E} may each independently be a straight chained alkyl group having the chemical formula —(CH₂)ₓCH₃, where x is from 0 to 17, such as 0 (a methyl group), 1, 2, 3, 4, 5, 6, 7, 8, 9, 10, 11, 12, 13, 14, 15, 16, 17, or 18 carbon atoms. In additional embodiments, R¹, R_{A}, R_{B}, R_{C}, R_{D}, and R_{E} may each independently be branched alkyl groups having from 3 to 18 carbon atoms, such as 3, 4, 5, 6, 7, 8, 9, 10, 11, 12, 13, 14, 15, 16, 17, or 18 carbon atoms. In some embodiments, the alkyl group may include a ring structure, such as a pentane ring, a hexane ring, etc.

In some embodiments, R¹, R_{A}, R_{B}, R_{C}, R_{D}, and R_{E} may each independently comprise a C₁-C₁₈ hydroxyl alkyl group. The term “hydroxyl alkyl group” refers to a functional group that includes one or more a hydroxyl moiety (—OH) bonded to an alkyl group. According to embodiments, the hydroxyl alkyl group may include 1, 2, 3, 4, 5, or even more hydroxyl moieties. In some embodiments, R¹, R_{A}, R_{B}, R_{C}, R_{D}, and R_{E} may each independently be a straight chained hydroxyl alkyl group having the chemical formula —(CH₂)ₓOH, where x is from 1 to 18. In additional embodiments, R¹, R_{A}, R_{B}, R_{C}, R_{D}, and R_{E} may each independently be branched hydroxyl alkyl groups having from 1 to 18 carbon atoms and at least one hydroxyl group.

In some embodiments, R¹, R_{A}, R_{B}, R_{C}, R_{D}, and R_{E} may each independently comprise a C₁-C₁₈ alkenyl group. The term “alkenyl group” refers to a functional group consisting of hydrogen and carbon atoms where at least two carbon atoms have a double bond. In some embodiments, the alkenyl group may have a single carbon to carbon double bond that is at the end of moiety (i.e., having the structure —(CH₂)ₓCH═CH₂, where x is from 0 to 16, such as 0, 1, 2, 3, 4, 5, 6, 7, 8, 9, 10, 11, 12, 13, 14, 15, or 16).

In some embodiments, R¹, R_{A}, R_{B}, R_{C}, R_{D}, and R_{E} may each independently comprise a C₁-C₁₈ alkynyl group. The term “alkynyl group” refers to a functional group consisting of hydrogen and carbon atoms where at least two carbon atoms have a triple bond. In some embodiments, R_{A}, R_{B}, R_{C}, R_{D}, and R_{E} may each independently have a single carbon to carbon triple bond that is at the end of moiety (i.e., having the structure —(CH₂)ₓC≡CH, where x is from 0 to 16, such as 0, 1, 2, 3, 4, 5, 6, 7, 8, 9, 10, 11, 12, 13, 14, 15, or 16).

In some embodiments, R¹, R_{A}, R_{B}, R_{C}, R_{D}, and R_{E} may each independently comprise one or more of a carbon-carbon double bond, a carbon-carbon triple bond, or a combination thereof, provided that R¹ does not comprise a terminal alkyne.

In some embodiments, R¹, R_{A}, R_{B}, R_{C}, R_{D}, and R_{E} may each independently be a C₁-C₁₈ acryl group. The term “acryl group” refers to a functional group consisting of a carbon-carbon double bond and a carbon-oxygen double bond separated by a carbon-carbon single bond. The acryl group may have the general formula —(CH₂)ₙCOCHCH₂, where n is any integer from 0 to 15, such as 0, 1, 2, 3, 4, 5, 6, 7, 8, 9, 10, 11, 12, 13, 14, or 15.

In some embodiments, R¹, R_{A}, R_{B}, R_{C}, R_{D}, and R_{E} may each independently be a C₁-C₁₈ functional alkyl group. The term “functional alkyl group” refers to an alkyl group which includes at least one moiety bonded to any carbon atom of the alkyl group. In some embodiments, the functional alkyl group may comprise more than one of the same moiety. In some embodiments, the functional alkyl group may comprise two or more different moieties. In some embodiments, the functional alkyl group may comprise a moiety chosen form a carboxyl group (i.e., —COOH), an amine group (i.e., —NH₂), or a thiol group (i.e., —SH).

In some embodiments, R¹ may be a C₂-C₁₇ alkyl group, and R_{A}, R_{B}, R_{C}, R_{D}, and R_{E} may each be hydrogen. For example, R¹ may be a C₄-C₁₆ alkyl group, a C₆-C₁₄ alkyl group, or a C₈-C₁₂ alkyl group. In some embodiments, R¹ may be a C₁-C₁₇, a C₁-C₁₆, a C₁-C₁₈, a C₁-C₁₄, a C₁-C₁₃, a C₁-C₁₂, a C₁-C₁₁, a C₁-C₁₀, a C₁-C₉, a C₁-C₈, a C₁-C₇, a C₁-C₆, a C₁-C₅, a C₁-C₄, a C₁-C₃, or a C₁-C₂ alkyl group. In some embodiments, R¹ may be a C₂-C₁₈, C₃-C₁₈, C₄-C₁₈, C₅-C₁₈, C₆-C₁₈, C₇-C₁₈, C₈-C₁₈, C₉-C₁₈, C₁₀-C₁₈, C₁₁-C₁₈, C₁₂-C₁₈, C₁₃-C₁₈, C₁₄-C₁₈, C₁₅-C₁₈, C₁₆-C₁₈, or C₁₇-C₁₈ alkyl group. In one embodiment, R¹ may be a C₁₀ alkyl group (i.e., a decyl group) and R_{A}, R_{B}, R_{C}, R_{D}, and R_{E} may each be hydrogen. In one embodiment, R¹ may be a C₁₂ alkyl group (i.e., a dodecyl group) and R_{A}, R_{B}, R_{C}, R_{D}, and R_{E} may each be hydrogen.

As described herein, in Chemical Structure #1, one or both of X may be —SH or Y may be —S—. For example, in some embodiments, X is —SH and Y is —S—, as is depicted in Chemical Structure #2.

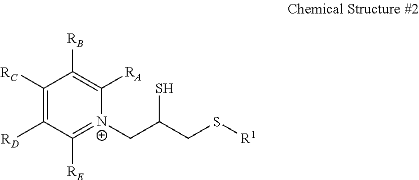

According to additional embodiments, one of X is not —SH or Y is not —S—. For example, in some embodiments, X is —SH and Y is —CH₂, —NH, or —O—. Chemical Structure #3 depicts an embodiment where X is —SH and Y is —O—. Chemical Structure #4 depicts an embodiment where X is —SH and Y is —CH₂ encompassed in the R¹ group. Chemical Structure

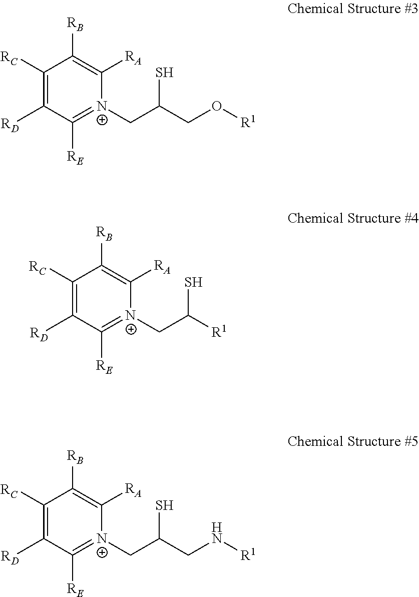

In additional embodiments, Y is —S— and X is not —SH. For example, in some embodiments, Y is —S— and X is —CH₃, —NH₂, or —OH. Chemical Structure #6 depicts an embodiment where Y is —S— and X is —CH₃. Chemical Structure #7 depicts an embodiment where where Y is —S— and X is —NH₂. Chemical Structure #8 depicts an embodiment where Y is —S— and X is —OH.

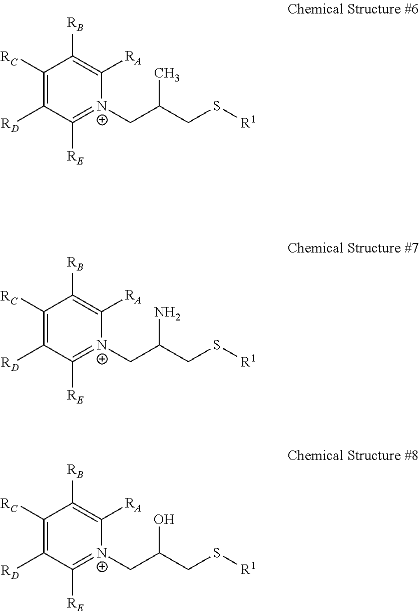

According to additional embodiments, any one or a mixture of the pyridinium-based compounds described herein may be utilized in a method for preventing corrosion and/or may be present in a material that may be utilized for preventing corrosion. In any of these materials and methods, one species or a combination of species of the pyridinium-based compounds may be utilized. The described materials and/or methods may include any pyridinium-based compound described herein, including without limitation those of Chemical Structures #1-#8.

According to one or more embodiments, it is contemplated that the presently disclosed compounds may be fabricated using organic chemistry techniques known to those skilled in the art. Without limitation, in some embodiments, the corrosion resistant compounds may be fabricated by grafting a functional group (such as that including the X and Y atoms) onto a pyridinium compound. For example, if X is —OH, a substituted epoxide may be reacted with pyridinium, according to Reaction Scheme #1. For example, if X is —SH, a substituted thiirane may be reacted with pyridinium, according to Reaction Scheme #2.

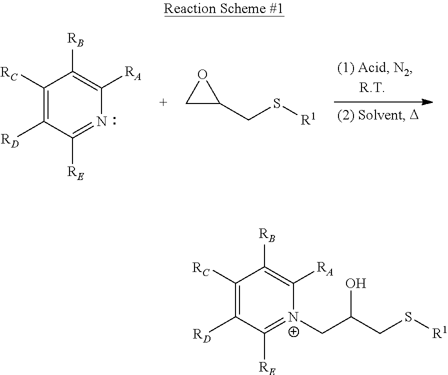

In Reaction Scheme #1, pyridine and acid (e.g. hydrochloric acid or hydrobromic acid) are mixed and purged with nitrogen at room temperature, followed by adding solvent (e.g. monoethylene glycol (MEG), diethylene glycol monoethyl ether (DGME) or 2-butoxyethanol) and then substituted epoxide (e.g. 1-methylthio-2,3-epoxypropane). The reaction is carried out an elevated temperature such as from 65° C. to 110° C.

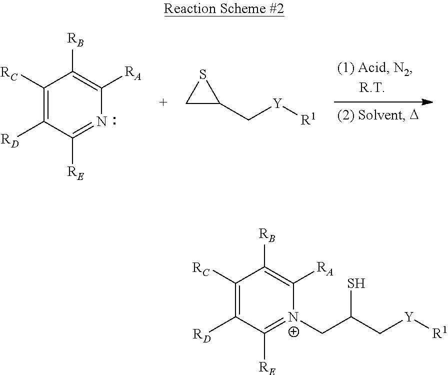

In Reaction Scheme #2, pyridine and acid (e.g. hydrochloric acid or hydrobromic acid) are mixed and purged with nitrogen at room temperature, followed by adding solvent (e.g. monoethylene glycol (MEG), diethylene glycol monoethyl ether (DGME), or 2-butoxyethanol) and then substituted thiirane (e.g. 2-(methoxymethyl) thiirane). The reaction is carried out at an elevated temperature such as from 65° C. to 110° C.

In one or more embodiments, the pyridinium-based compounds may be used to mitigate corrosion of a material. In some embodiments, the pyridinium-based compounds may be used to mitigate corrosion of a metallic surface, such as a metallic surface in a wet environment. According to one or more embodiments, the pyridinium-based compounds may be included in a well fluid to mitigate the corrosion of surfaces exposed to the well fluid. Thus, a well fluid may include the pyridinium-based compounds at a concentration by weight of 0.1 parts per million to 100 parts per million, 0.2 parts per million to 50 parts per million, 0.2 parts per million to 40 parts per million, 0.2 parts per million to 30 parts per million, 0.2 parts per million to 20 parts per million, 0.2 parts per million to 10 parts per million, 0.3 parts per million to 8 parts per million, 0.4 parts per million to 6 parts per million, 0.5 parts per million to 5 parts per million, 0.5 parts per million to 2 parts per million, or 1 part per million to 2 parts per million of the well fluid. In embodiments, the well fluid may further comprise petroleum hydrocarbons.

In embodiments, a process for mitigating corrosion of a metallic surface may include contacting the metallic surface with a solution comprising the pyridinium-based compounds at a concentration by weight of 0.1 parts per million to 100 parts per million, 0.2 parts per million to 50 parts per million, 0.2 parts per million to 40 parts per million, 0.2 parts per million to 30 parts per million, 0.2 parts per million to 20 parts per million, 0.2 parts per million to 10 parts per million, 0.3 parts per million to 8 parts per million, 0.4 parts per million to 6 parts per million, 0.5 parts per million to 5 parts per million, 0.5 parts per million to 2 parts per million, or 1 part per million to 2 parts per million of the well fluid. According to embodiments, the metallic surface comprises steel. In some embodiments, the metallic surface comprises carbon steel. In embodiments, the metallic surface may be contacted with one or more pyridinium-based compounds at least one time, at least two times, at least three times, at least four times, or even at least five times. It should be understood that when the metallic surface is contacted with the one or more pyridinium-based compounds multiple times, each contacting may be accomplished using the same or different concentrations of the compound described herein.

In some embodiments, the one or more pyridinium-based compounds described herein may be used as an active component in a formulation for inhibiting corrosion. According to one or more embodiments, a corrosion inhibitor formulation comprises the one or more pyridinium-based compounds and water. In some embodiments, the corrosion inhibitor formulation further comprises a synergist, a surfactant, a supporting component, a secondary solvent, a coupling agent, an ethoxylated amine, or a combination thereof.

According to one or more embodiments, the one or more pyridinium-based compounds may be present in the corrosion inhibitor formulation at a concentration of from 0.1 to 50 weight percent, 0.2 to 40 weight percent, 0.3 to 40 weight percent, 0.5 to 40 weight percent, 1 to 40 weight percent, 2 to 40 weight percent, 3 to 40 weight percent, 4 to 40 weight percent, 5 to 40 weight percent, 5 to 35 weight percent, 5 to 30 weight percent, 5 to 25 weight percent, 5 to 20 weight percent, or 10 to 20 weight percent. In embodiments, water may be present in the corrosion inhibitor formulation at a concentration of from 20 to 99.9 weight percent, 20 to 99 weight percent, 20 to 95 weight percent, 20 to 90 weight percent, 30 to 90 weight percent, 40 to 90 weight percent, 50 to 90 weight percent, 50 to 80 weight percent, 60 to 80 weight percent, or 65 to 75 weight percent.

In some embodiments, the corrosion inhibitor formulation includes a synergist. The synergist may act to facilitate the adsorption of an active component comprising a quaternary ammonium or pyridinium substituent onto the surface of a metal. Without being bound by theory, it is believed that the synergist adsorbs onto a metal surface while also attracting an active component comprising a quaternary ammonium or pyridinium substituent. In some embodiments, the synergist comprises a thiol substituent. In other embodiments, the synergist is an alkyl iodide. According to one or more embodiments, the synergist is thioglycolic acid, 2-mercaptoethanol, or a combination thereof. In embodiments, the synergist is present in the corrosion inhibitor formulation at a concentration of from 0.1 to 20 weight percent, 0.2 to 20 weight percent, 0.3 to 20 weight percent, 0.5 to 20 weight percent, 1 to 20 weight percent, 1 to 15 weight percent, 1 to 10 weight percent, 2 to 10 weight percent, 2 to 8 weight percent, or 2 to 6 weight percent.

In embodiments, the corrosion inhibitor formulation includes a secondary solvent. The secondary solvent may act to clean the metal for adsorption of an active component. In embodiments, the secondary solvent comprises ethylene glycol, ethylene diamine, or combinations thereof. According to one or more embodiments, the secondary solvent is present in the corrosion inhibitor formulation at a concentration of from 0.1 to 20 weight percent, 0.2 to 20 weight percent, 0.3 to 20 weight percent, 0.5 to 20 weight percent, 1 to 20 weight percent, 1 to 15 weight percent, 1 to 10 weight percent, 1 to 8 weight percent, 1 to 6 weight percent, or 2 to 5 weight percent.

According to one or more embodiments, the corrosion inhibitor formulation may include a surfactant. In embodiments, the surfactant may comprise an ethoxylated alcohol. According to one or more embodiments, the ethoxylated alcohol may comprise a carbon chain length of C₅ to C₂₀, C₈ to C₁₈, C₁₀ to C₁₅, or C₁₂ to C₁₄. According to one or more embodiments, the surfactant may be present in the corrosion inhibitor formulation at a concentration of from 0.1 to 10 weight percent, 0.1 to 5 weight percent, 0.1 to 2 weight percent, 0.2 to 2 weight percent, 0.2 to 1.5 weight percent, 0.2 to 1 weight percent, 0.2 to 0.8 weight percent, 0.2 to 0.6 weight percent, 0.3 to 0.6 weight percent, or 0.4 to 0.6 weight percent.

In embodiments, the corrosion inhibitor formulation may comprise a coupling agent. The coupling agent may act to mitigate phase separation of components in the corrosion inhibitor formulation. The risk of phase separation may be especially acute in environments with a large temperature range. According to one or more embodiments, the coupling agent may be an alkyl imino dipropionic acid sodium salt. In embodiments, the coupling agent may be present in the corrosion inhibitor formulation at a concentration of from 0.1 to 10 weight percent, 0.1 to 5 weight percent, 0.2 to 5 weight percent, 0.3 to 5 weight percent, 0.3 to 3 weight percent, 0.3 to 2 weight percent, 0.5 to 2 weight percent, 0.5 to 1.5 weight percent, 0.7 to 1.5 weight percent, or 0.8 to 1.2 weight percent.

In some embodiments, the corrosion inhibitor formulation may comprise an ethoxylated amine. The ethoxylated amine may facilitate film formation and neutralize acids present in the environment. In embodiments, the ethoxylated amine may be present in the corrosion inhibitor formulation at a concentration of from 0.1 to 10 weight percent, 0.1 to 5 weight percent, 0.2 to 5 weight percent, 0.3 to 5 weight percent, 0.3 to 3 weight percent, 0.3 to 2 weight percent, 0.5 to 2 weight percent, 0.5 to 1.5 weight percent, 0.7 to 1.5 weight percent, or 0.8 to 1.2 weight percent.

According to one or more embodiments, the corrosion inhibitor formulation may include a supporting component. The supporting component may act cooperatively, along with the active component, to mitigate corrosion. In some embodiments, the supporting component mitigates sweet corrosion in wet environments containing both CO₂ and H₂S. According to one or more embodiments, the supporting component may comprise imidazoline. In embodiments, imidazoline may be present in the corrosion inhibitor formulation at a concentration of from 0.1 to 20 weight percent, 0.2 to 20 weight percent, 0.3 to 20 weight percent, 0.5 to 20 weight percent, 1 to 20 weight percent, 1 to 15 weight percent, 1 to 10 weight percent, 2 to 10 weight percent, 2 to 8 weight percent, or 2 to 6 weight percent.

In embodiments, a process for inhibiting corrosion may comprise contacting a metallic surface with a corrosion inhibitor formulation according to embodiments described herein. According to embodiments, the metallic surface comprises steel. In some embodiments, the metallic surface comprises carbon steel. In embodiments, the metallic surface may be contacted with a solution containing the corrosion inhibitor formulation at a concentration by weight of 0.5 parts per million to 500 parts per million, 0.5 parts per million to 200 parts per million, 0.5 parts per million to 100 parts per million, 0.5 parts per million to 50 parts per million, 1 part per million to 50 parts per million, 1 part per million to 40 parts per million, 2 part per million to 40 parts per million, 2 part per million to 30 parts per million, 3 part per million to 30 parts per million, 3 part per million to 20 parts per million, 5 part per million to 20 parts per million, or 5 part per million to 15 parts per million. In embodiments, the metallic surface may be contacted with the corrosion inhibitor formulation at least one time, at least two times, at least three times, at least four times, or even at least five times. It should be understood that when the metallic surface is contacted with the corrosion inhibitor formulation multiple times, each contacting may be accomplished using the same or different concentrations of the compound described herein.

Many oil and gas processing facilities such as gas oil separation plants and pipelines include metallic surfaces exposed to well fluids. Thus, adding the corrosion inhibitor formulation described herein to the well fluids may mitigate corrosion of the metallic surfaces. Thus, according to one or more embodiments, a process for inhibiting corrosion includes adding the corrosion inhibitor formulation described herein to a well fluid to effect a concentration by weight of the formulation in the well fluid of from 0.5 parts per million to 500 parts per million, 0.5 parts per million to 200 parts per million, 0.5 parts per million to 100 parts per million, 0.5 parts per million to 50 parts per million, 1 part per million to 50 parts per million, 1 part per million to 40 parts per million, 2 part per million to 40 parts per million, 2 part per million to 30 parts per million, 3 part per million to 30 parts per million, 3 part per million to 20 parts per million, 5 part per million to 20 parts per million, or 5 part per million to 15 parts per million.

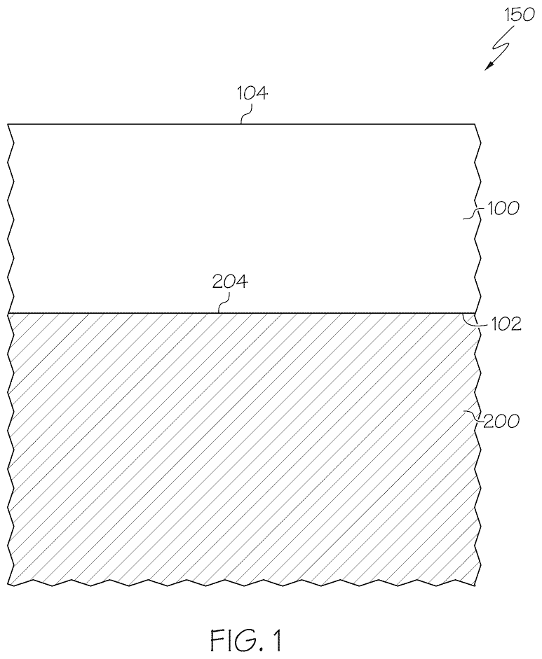

Referring now to FIG. 1, according to one or more embodiments, the corrosion-resistant substrates 150 may comprise a substrate 200 that may comprise at least a first surface 204. The term “substrate” may refer to any object with at least one surface where a solution may contact and form a film that remains on at least a portion of that surface. The corrosion-resistant substrates 150 may also comprise a corrosion-resistant film 100 that comprises at least a first surface 102 and a second surface 104 opposite the first surface 102. The corrosion-resistant film 100 may be positioned on at least a portion of the first surface 204 of the substrate 200. As depicted, the corrosion-resistant substrates 150 may have the first surface 102 of the corrosion-resistant film 100 positioned on and in direct contact with at least a portion of the first surface 204 of the substrate 200. The second surface 104 of the corrosion-resistant film 100 may be an “air-side” surface defining the outer edge of the corrosion-resistant substrate 150.

In one or more embodiments, the corrosion-resistant film 100 may be a solid. The term “solid” may refer to a material that is generally firm, stable in shape, and is not a liquid or a fluid. Accordingly, when the corrosion-resistant film 100 is a solid, the first surface 102 of the corrosion-resistant film 100 adheres to the first surface 204 of the substrate 200 so that the corrosion-resistant film 100 remains on the substrate 200 and holds its shape while the substrate 200 and/or the corrosion-resistant film 100 is moved.

In one or more embodiments, the corrosion-resistant film 100 has a thickness of from 0.1 nm to 1,000 nm, such as a thickness of from 0.1 nm to 900 nm, from 0.1 nm to 800 nm, from 0.1 nm to 700 nm, from 0.1 nm to 600 nm, from 0.1 nm to 500 nm, from 0.1 nm to 400 nm, from 0.1 nm to 300 nm, from 0.1 nm to 200 nm, from 0.1 nm to 100 nm, from 1 nm to 1,000 nm, from 10 nm to 1,000 nm, from 50 nm to 1,000 nm, from 100 nm to 1,000 nm, from 200 nm to 1,000 nm, from 300 nm to 1,000 nm, from 400 nm to 1,000 nm, from 500 nm to 1,000 nm, from 600 nm to 1,000 nm, from 700 nm to 1,000 nm, from 800 nm to 1,000 nm, from 900 nm to 1,000 nm, from 10 nm to 900 nm, from 100 nm to 800 nm, from 200 nm to 700 nm, or from 300 nm to 600 nm.

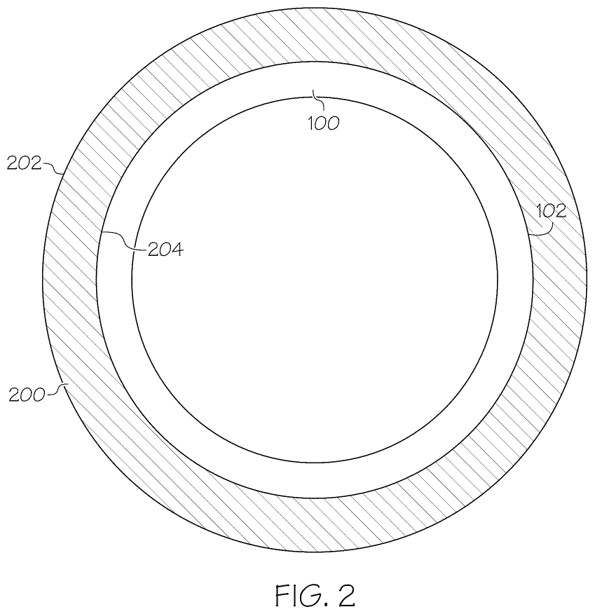

Now, referring to FIG. 2, in one or more embodiments, the substrate 200 of the corrosion-resistant substrates may be a metal pipe that comprises at least a first surface 204 and a second surface 202. The term “pipe” may refer to a tubular hollow cylinder having a circular, or near circular, cross section that is used to transport substances (for example liquids, gases, slurries, powders, small solids, etc.). The metal pipe may comprise one or more metals and one or more surfaces of the metal pipe may comprise metal oxides. For example, the metal pipe may comprise carbon steel. In some embodiments, the first surface 204 of the metal pipe may be the internal surface of the metal pipe, and the pipe may further comprise an outer surface 202. The term “internal surface” may refer to the surface of the inside of the metal pipe that is enclosed within the tubular cylinder of the metal pipe. For example, when the substrate 200 is a metal pipe and the first surface 204 is the internal surface of the metal pipe, the first surface 102 of the corrosion-resistant film 100 may be in direct contact with the internal surface of the metal pipe. Without being bound by a theory, it is believed that the corrosion-resistant film 100 being in direct contact with a least a portion of the internal surface of the metal pipe creates a barrier between the substances that flow through the metal pipe and the internal surface of the metal pipe.

The present disclosure is also directed to methods of producing corrosion-resistant substrates 150 and various embodiments of corrosion inhibitor solutions. The methods of producing corrosion-resistant substrates 150 may comprise contacting at least a portion of a first surface 204 of a substrate 200 with a corrosion inhibitor solution, where the corrosion inhibitor solution comprises a solvent and one or more pyridinium-based compounds. Then, the methods may further comprise drying the corrosion inhibitor solution to produce the corrosion-resistant film 100 on the substrate 200, where at least a portion of the solvent is expelled from the corrosion inhibitor solution during the drying to form the solid corrosion-resistant film 100. For example, when the substrate 200 is a pipe and the first surface 204 is the internal surface of the pipe, the corrosion inhibitor solution is adhered on the internal surface of the pipe and the corrosion inhibitor solution dries on the internal surface of the pipe to form the solid corrosion-resistant film 100 on the internal surface of the pipe.

In one or more embodiments, the corrosion inhibitor solution may comprise a solvent and the pyridinium-based compounds, as described herein. In some embodiments, the solvent may comprise water, an alcohol, aromatic naphtha, or combinations thereof.

According to one or more embodiments, the corrosion inhibitor solution may comprise from 1 wt. % to 50 wt. % of the pyridinium-based compounds. In some embodiments, the corrosion inhibitor solution may comprise from 1 wt. % to 45 wt. %, from 5 wt. % to 40 wt. %, from 10 wt. % to 35 wt. %, or from 15 wt. % to 30 wt. %, or 15 wt. % to 25 wt. % of the pyridinium-based compounds. In some embodiments, the corrosion inhibitor solution may comprise from 5 wt. % to 50 wt. %, 5 wt. % to 40 wt. %, 5 wt. % to 35 wt. %, 5 wt. % to 30 wt. %, 5 wt. % to 25 wt. %, 5 wt. % to 20 wt. %, 5 wt. % to 15 wt. %, or 5 wt. % to 10 wt. %, of the pyridinium-based compounds.

In some embodiments, drying the corrosion inhibitor solution in order to produce the solid corrosion-resistant film 100 may include passively drying the corrosion inhibitor solution. Passively drying the corrosion inhibitor solution may refer to allowing the corrosion inhibitor solution to dry on the first surface 204 of the substrate 200 without the use of an external heat source. For example, the corrosion inhibitor solution may be allowed to dry at room temperature after contacting the first surface 204 of the substrate 200 or any similar method where the corrosion inhibitor solution is not heated with an external heat source. Further, drying the corrosion inhibitor solution in order to produce the solid corrosion-resistant film 100 may include heating the corrosion inhibitor solution with a heat source. Heating the corrosion inhibitor solution with a heat source may refer to any method where heat from an outside source is transferred to the corrosion inhibitor solution and the first surface 204 of the substrate 200 in order to dry the corrosion inhibitor solution. For example, a heat lamp may be directed onto the corrosion inhibitor solution and the first surface 204 of the substrate 200 in order to accelerate the drying time of the corrosion inhibitor solution. In another example, external processing units, liquids, or gases may transfer heat to the corrosion inhibitor solution and the first surface 204 of the substrate 200 in order to accelerate the drying time of the corrosion inhibitor solution. In some embodiments, the drying of the corrosion inhibitor solution in order to produce the solid corrosion-resistant film 100 may include both passively drying the corrosion inhibitor solution and heating the corrosion inhibitor solution with a heat source.

According to one or more additional embodiments of the present disclosure, subsurface formations may be acidized, where the pyridinium-based compounds may be utilized to reduce corrosion of downhole metal surfaces, such as steel tubing.

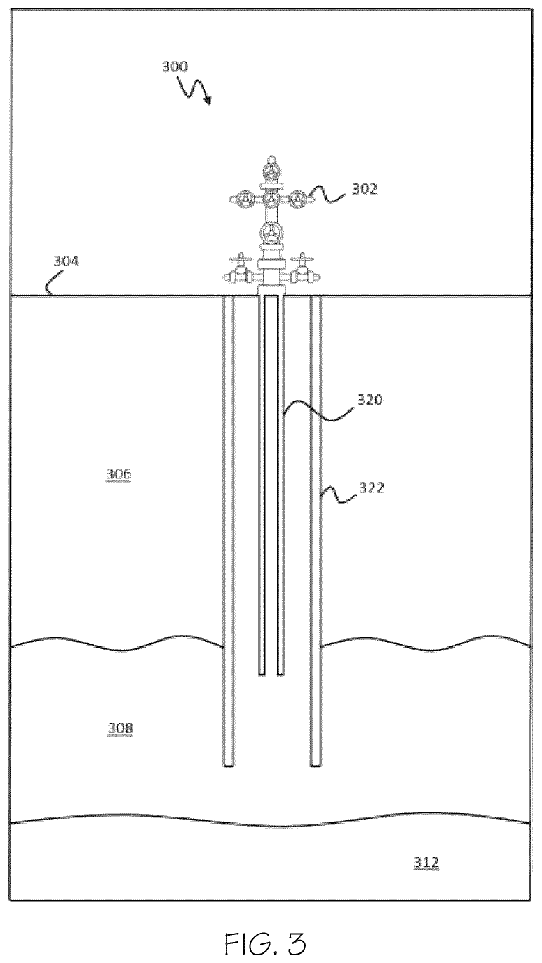

Referring now to FIG. 3, a hydrocarbon extraction well 300 is schematically depicted, where the downhole region is a cross-sectional view through a diameter of the casting 322 and tubing 320. It should be understood that FIG. 3 is a simplified depiction of a hydrocarbon extraction well 300, and other non-depicted components on a hydrocarbon extraction well 300 may be present in real-world operation. The hydrocarbon extraction well 300 may extend from the surface 304, through a subterranean zone 306, and into a subsurface formation 308 that contains extractable hydrocarbons such as oil and/or gas. Subsurface zone 312 represents another subsurface formation that does not necessarily include extractable hydrocarbons. In general, the casing 322 may form a passage to the subsurface formation 308, where cementing may be present between the natural formation and the casing 322 (not shown in FIG. 3). A wellhead 302 may connect to tubing 320 that passes through the interior of the casing 322. In general, during hydrocarbon extraction, the hydrocarbons may flow upwards through the tubing 320 and to the wellhead 302. The tubing 320 may be metal in material, such as steel. In some embodiments, the tubing 320 may be carbon steel 320.

According to embodiments, acidizing treatments may be employed as a stimulation process intended to enlarge the natural pores in material such as rock in the subsurface formation 308 and, thus, facilitate the flow of hydrocarbons that results in an increased productivity for oil and/or gas producing facilities. In general, during acidizing treatments, an acidic treatment fluid may be injected into the subsurface formation 308 from the surface 304 through the tubing 320. The acidic treatment fluid used for such application may be highly acidic and may generally cause the tubing 320 through which it passes to corrode. As such, at least a portion of the tubing 322 (such as the interior surface) is exposed to an acidic environment during acidizing treatments.

According to one or more embodiments, the acidic treatment fluid may comprise one or more acids. A wide range of acids are contemplated as suitable in the processes described herein, and an acid may be selected based on the type of rock that is to be treated and/or the intended goals of the acidizing treatment. Without limitation, according to some embodiments, the one or more acids in the acid treatment fluid may be chosen from hydrochloric acid, formic acid, acetic acid hydrofluoric acid, nitric acid, sulfuric acid, hydrobromic acid, hydroiodic acid, perchloric acid, or chloric acid.

According to embodiments, the environment in which the tubing 322 experiences is acidic and may be extremely acidic. For example, according to one or more embodiments, the acidic environment in contact with the tubing 322 may have a pH of 6 or less. In additional embodiments, the pH of the acidic environment may be less than or equal to 5.5, less than or equal to 5, less than or equal to 4.5, less than or equal to 4, less than or equal to 3.5, less than or equal to 3, less than or equal to 2.5, or even less than or equal to 2. Generally, the composition of the acidic treatment fluid and the amount used for acidizing treatment may determine the downhole acidity.

In general, the downhole conditions of the acidic environment that is contacting at least a portion of the tubing 322 may be relatively high temperature and/or relatively high pressure (sometimes referred to in industry as “HTHP”). For example, the acidic environment may have a temperature of at least 50° C., at least 100° C., at least 150° C., at least 200° C., at least 250° C., or even at least 300° C., in some embodiments up to 1000° C. The pressure of the acidic environment may be at least 250 psi, at least 500 psi, at least 750 psi, at least 1000 psi, at least 2000 psi, at least 3000 psi, at least 4000 psi, at least 5000 psi, at least 7500 psi, or even at least 10,000 psi, in some embodiments up to 30,000 psi. These HTHP conditions may degrade conventional pyridinium-based compounds. However, without being bound by theory, it is believed that the presently disclosed pyridinium-based compounds may not degrade or may degrade to a lesser degree at such temperatures and pressures as compared to conventional corrosion inhibitors.

According to embodiments, during acidizing, at least a portion of the tubing 322 may be contacted by one or more pyridinium-based compounds. In some embodiments, the pyridinium-based compound may be mixed into the acidic treatment fluid (or be a portion of the acidic treatment fluid). In other embodiments, the pyridinium-based compounds may be present in a solvent or carrier fluid and be injected before, after, or concurrently with the acidic treatment fluid.

Without being bound by theory, in some embodiments, it is believed that the pyridinium-based compound may form a film on the surface of the tubing 322, which may prevent corrosion. In other embodiments, a measurable film layer may not be formed, but corrosion on the tubing may none the less be mitigated. The amount of pyridinium-based compound utilized may be sufficient to mitigate corrosion, but is not particularly limiting in the embodiments described herein. For example, from several ppm (e.g., at least 2 ppm, at least 5 ppm, at least 25 ppm, at least 50 ppm, at least 100 ppm, at least 500 ppm, or even at least 1000 ppm) of pyridinium-based compound up to at least 1 wt. %, at least 5 wt. %, or even at least 10 wt. % of the pyridinium-based compound may be present in the acidic environment. In some embodiments, the amount of pyridinium-based compound may depend on the strength of the acid.

The subject matter of the present disclosure has been described in detail and by reference to specific embodiments. It should be understood that any detailed description of a component or feature of an embodiment does not necessarily imply that the component or feature is essential to the particular embodiment or to any other embodiment. Further, it should be apparent to those skilled in the art that various modifications and variations can be made to the described embodiments without departing from the spirit and scope of the claimed subject matter.

Numerous aspects of the present application are described hereinbelow as Aspects 1-20.

Aspect 1. A pyridinium-based compound, the compound having the structure of Chemical Structure #1 or a salt thereof, wherein: one or both of X is —SH or Y is —S—; R¹ is a C₁-C₁₈ alkyl group, a C₁-C₁₈ hydroxyl alkyl group, a C₁-C₁₈ alkenyl group, a C₁-C₁₈ internal alkynyl group, a C₁-C₁₈ acryl group, a C₁-C₁₈ cycloalkyl group, or a C₁-C₁₈ functional alkyl group; and R_{A}, R_{B}, R_{C}, R_{D}, and R_{E} are each independently chosen from hydrogen, a C₁-C₁₈ alkyl group, a C₁-C₁₈ hydroxyl alkyl group, a C₁-C₁₈ alkenyl group, a C₁-C₁₈ alkynyl group, a C₁-C₁₈ acryl group, a C₁-C₁₈ cycloalkyl group, or a C₁-C₁₈ functional alkyl group.

Aspect 2. The pyridinium-based compound of aspect 1, wherein X is —SH.

Aspect 3. The pyridinium-based compound of aspects 1 or 2, wherein R_{A}, R_{B}, R_{C}, R_{D}, and R_{E} are each hydrogen.

Aspect 4. The pyridinium-based compound of any one of aspects 1 to 3, wherein the C₁-C₁₈ functional alkyl group comprises a moiety chosen from a carboxyl group, an amine group, or a thiol group.

Aspect 5. The pyridinium-based compound of any one of aspects 1 to 4, wherein R¹ is a C₁-C₁₈ alkyl group.

Aspect 6. The pyridinium-based compound of any one of aspects 1 to 5, wherein Y is —S—.

Aspect 7. The pyridinium-based compound of aspect 1, wherein X is —SH and Y is —S—.

Aspect 8. A material comprising the pyridinium-based compound of any one of aspects 1 to 7.

Aspect 9. The material of aspect 8, wherein the material is a well fluid comprising the pyridinium-based compound of any one of aspects 1 to 7 at a concentration by weight that is greater than or equal to 0.1 parts per million and less than or equal to 100 parts per million of the well fluid.

Aspect 10. The material of aspects 8 or 9, wherein the material is a well fluid comprising the pyridinium-based compound of any one of aspects 1 to 7 at a concentration by weight that is greater than or equal to 0.2 parts per million and less than or equal to 20 parts per million of the well fluid.

Aspect 11. The material of aspects 9 or 10, wherein the well fluid further comprises petroleum hydrocarbons.

Aspect 12. The material of aspect 8, wherein the material is a formulation for inhibiting corrosion comprising: the pyridinium-based compound of any one of aspects 1 to 7; and water.

Aspect 13. The material of aspect 12, further comprising one or more synergists, a nonionic surfactant, imidazoline, a secondary solvent, a coupling agent, and an ethoxylated amine.

Aspect 14. A method for inhibiting corrosion comprising contacting a metallic surface with the formulation of aspects 12 or 13.

Aspect 15. The method of aspect 14, wherein the formulation of aspects 12 or 13 comprises the pyridinium-based compound of any one of aspects 1 to 7 at a weight percentage that is greater than or equal to 1.0 wt % and less than or equal to 50 wt %.

Aspect 16. A corrosion-resistant substrate comprising: a substrate comprising a first surface; and a corrosion-resistant film positioned on at least a portion of the first surface of the substrate, wherein the corrosion-resistant film is solid, and wherein the corrosion-resistant film comprises the pyridinium-based compound of any one of aspects 1 to 7.

Aspect 17. The corrosion-resistant substrate of aspect 16, wherein the first surface is metal or metal oxide.

Aspect 18. The corrosion-resistant substrate of aspect 16, wherein the substrate is a metal pipe and the first surface is an internal surface of the metal pipe.

Aspect 19. A method of acidizing a subsurface formation, the method comprising: injecting an acidic treatment fluid into a subsurface formation through a tubing of an extraction well, thereby exposing at least a portion of the tubing to an acidic environment; wherein at least a portion of the tubing is contacted by the pyridinium-based compound of any one of aspects 1 to 7.

Aspect 20. The method of aspect 19, wherein the acidic treatment fluid comprises one or more acids chosen from hydrochloric acid, formic acid, acetic acid hydrofluoric acid, nitric acid, sulfuric acid, hydrobromic acid, hydroiodic acid, perchloric acid, or chloric acid, or combinations thereof.

### EXAMPLES

Examples are provided herein which may disclose one or more embodiments of the present disclosure. However, the Examples should not be viewed as limiting on the claimed embodiments hereinafter provided.

### Example 1—Synthesis of 1-[3-(methylthio) 2-hydroxypropyl]pyridinium salt

Pyridine (1.5 mol) and hydrochloric acid/Hydrobromic acid (1 mol) were added to a round bottom flask and purged with nitrogen, and stirred at RT (25° C.) for 10 min. Then, 1 mole of solvent (MEG or DGME or 2-Butoxyethanol) was added, followed by adding alkyl epoxide (1-methylthio-2,3-epoxypropane) (1 mol), to the first flask or vessel and again stirred for 30 min and heated at 100° C. for 10 hours. At the end of elapsed time, sample was collected for ¹H-NMR analysis. At the end of experiment, pyridinium salt of epoxide (1-[3-(methylthio) 2-hydroxypropyl] pyridinium salt) in respective solvent was collected and additional analysis and characterization was carried out.

In case of water as solvent at the end sample was freeze dried remove the water. A general reaction scheme of 1-alkylthio-2,3-epoxypropane) with pyridine is shown in Reaction Scheme #3.

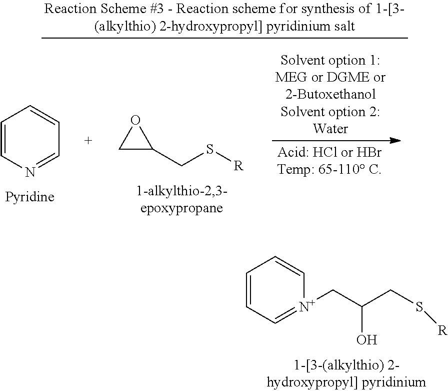

### Example 2—Synthesis of Thiirane Pyridinium Salt

Pyridine (1.5 mol) and hydrochloric acid or hydrobromic acid (1 mol) were added to a round bottom flask and purged with nitrogen, and stirred at room temperature (25° C.) for 10 min. Then, 1 mole of solvent (MEG or DGME or 2-butoxyethanol) was added, followed by adding 2-(Methoxymethyl) thiirane (1 mol), to the first flask or vessel and again stirred for 30 min and heated at 100° C. for 10 hours. At the end of the elapsed time, sample was collected for ¹H-NMR analysis. At the end of experiment, pyridinium salt of thiirane in respective solvent was collected and additional analysis and characterization was carried out.

In case of water was used as solvent, at the end of experiment, sample was freeze dried to remove the water. A general reaction scheme is shown in Reaction Scheme #4.

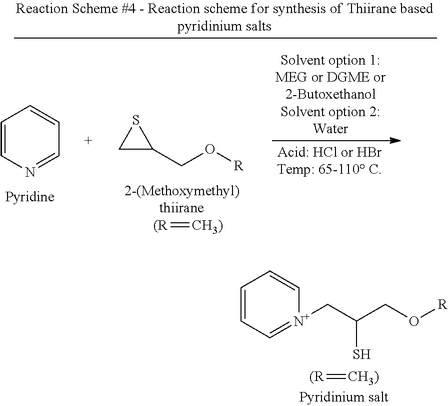

It is noted that one or more of the following claims utilize the term “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that where a first component is described as “comprising” a second component, it is contemplated that, in some embodiments, the first component “consists” or “consists essentially of” that second component. It should further be understood that where a first component is described as “comprising” a second component, it is contemplated that, in some embodiments, the first component comprises at least 10%, at least 20%, at least 30%, at least 40%, at least 50%, at least 60%, at least 70%, at least 80%, at least 90%, at least 95%, or even at least 99% that second component (where % can be weight % or molar %).

It is also noted that recitations herein of “at least one” component, element, etc., should not be used to create an inference that the alternative use of the articles “a” or “an” should be limited to a single component, element, etc.

For the purposes of describing and defining the presently disclosed technology it is noted that the terms “substantially” and “about” are utilized herein to represent the inherent degree of uncertainty that may be attributed to any quantitative comparison, value, measurement, or other representation. The terms “substantially” and “about” are also utilized herein to represent the degree by which a quantitative representation may vary from a stated reference without resulting in a change in the basic function of the subject matter at issue.

What is claimed is:

1. A pyridinium-based compound, the compound having the structure: 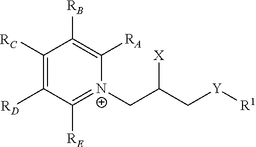
or a salt thereof, wherein:
one or both of X is —SH or Y is —S—;
R¹ is a C₁-C₁₈ alkyl group, a C₁-C₁₈ hydroxyl alkyl group, a C₁-C₁₈ alkenyl group, a C₁-C₁₈ internal alkynyl group, a C₁-C₁₈ acryl group, a C₁-C₁₈ cycloalkyl group, or a C₁-C₁₈ functional alkyl group; and
R_{A}, R_{B}, R_{C}, R_{D}, and R_{E} are each independently chosen from hydrogen, a C₁-C₁₈ alkyl group, a C₁-C₁₈ hydroxyl alkyl group, a C₁-C₁₈ alkenyl group, a C₁-C₁₈ alkynyl group, a C₁-C₁₈ acryl group, a C₁-C₁₈ cycloalkyl group, or a C₁-C₁₈ functional alkyl group.

2. The pyridinium-based compound of claim 1, wherein X is —SH.

3. The pyridinium-based compound of claim 1, wherein R_{A}, R_{B}, R_{C}, R_{D}, and R_{E} are each hydrogen.

4. The pyridinium-based compound of claim 1, wherein the C₁-C₁₈ functional alkyl group comprises a moiety chosen from a carboxyl group, an amine group, or a thiol group.

5. The pyridinium-based compound of claim 1, wherein R¹ is a C₁-C₁₈ alkyl group.

6. The pyridinium-based compound of claim 1, wherein Y is —S—.

7. The pyridinium-based compound of claim 1, wherein X is —SH and Y is —S—.

8. A material comprising the pyridinium-based compound of claim 1.

9. The material of claim 8, wherein the material is a well fluid comprising the pyridinium-based compound of claim 1 at a concentration by weight that is greater than or equal to 0.1 parts per million and less than or equal to 100 parts per million of the well fluid.

10. The material of claim 9, wherein the well fluid comprising the pyridinium-based compound of claim 1 at a concentration by weight that is greater than or equal to 0.2 parts per million and less than or equal to 20 parts per million of the well fluid.

11. The material of claim 9, wherein the well fluid further comprises petroleum hydrocarbons.

12. The material of claim 8, wherein the material is a formulation for inhibiting corrosion comprising:
the pyridinium-based compound of claim 1; and
water.

13. The material of claim 12, further comprising one or more of a synergist, a nonionic surfactant, imidazoline, a secondary solvent, a coupling agent, and an ethoxylated amine.

14. A method for inhibiting corrosion comprising contacting a metallic surface with the formulation of claim 12.

15. The method of claim 14, wherein the formulation of claim 12 comprises the pyridinium-based compound of claim 1 at a weight percentage that is greater than or equal to 1.0 wt % and less than or equal to 50 wt %.
